(19) United States
(12) Patent Application Publication
(10) Pub. No.: US 2024/0060967 A1
(43) Pub. Date: Feb. 22, 2024

(54) Urinary Tract Infection Alert/Detection Device and Method of Use Thereof

(71) Applicants: Seth Parker, Shelby, MI (US); Tracey Ryckman, Northville, MI (US)

(72) Inventors: Seth Parker, Shelby, MI (US); Tracey Ryckman, Northville, MI (US)

(21) Appl. No.: 18/161,418

(22) Filed: Jan. 30, 2023

Related U.S. Application Data
(60) Provisional application No. 63/399,283, filed on Aug. 19, 2022.

(51) Int. Cl. G01N 33/52; G01N 33/493

(52) U.S. Cl. CPC G01N 33/528; G01N 33/493

(57) ABSTRACT

The present invention relates to a non-invasive device for easily detecting a possible urinary tract infection (UTI). The device is in the form of a pad having a color indicating strip that includes detection reagents for detecting a UTI and an adhesive layer for releasably adhering the device inside an undergarment. The device is adhered on the crotch region such a few drops of urine are dropped onto the color indicating strip. The strip is configured to change color to one of green, yellow or orange, and red wherein the green color indicates absence of a UTI, the yellow or orange color indicates possibility of a UTI or dehydration, and the red color indicates definite UTI. The color indicating strip is configured to detect leukocytes, hemoglobin, and nitrates wherein the red color indicates at least 100,000 leukocytes in conformance with “McGreer Criteria” for a UTI.

### CROSS-REFERENCE TO RELATED APPLICATION

The present application claims priority to, and the benefit of, U.S. Provisional Application No. 63/399,283, which was filed on Aug. 19, 2022, and is incorporated herein by reference in its entirety.

### FIELD OF THE INVENTION

The present invention relates generally to the field of Unitary Tract Infection (UTI) detection devices and methods. More specifically, the present invention relates to a novel device for easily detecting UTI in a non-invasive manner. The device is designed as an undergarment liner and is releasably adhered on the crotch region inside an undergarment. A color strip of the device changes color when urine drops are dropped or placed thereon for indicating possibility of UTI. Accordingly, the present disclosure makes specific reference thereto. Nonetheless, it is to be appreciated that aspects of the present invention are also equally applicable to other like applications, devices, and methods of manufacture.

### BACKGROUND

By way of background, a urinary tract infection (UTI) is an infection in any part of the urinary system. The urinary system of a human includes the kidneys, ureters, bladder, and urethra. Most UTI infections involve the lower urinary tract that includes the bladder and the urethra. Research show that women are at greater risk of developing a UTI. When UTI is limited to the bladder of a person, the infection can be painful and annoying, with serious health problems resulting if left untreated. UTI's may occur without the classic symptoms such as burning sensations, pain and more causing the infection to go undetected.

Further, in such scenarios, the UTI may spread to the kidneys and can be more harmful. An untreated bladder infection may lead to a kidney or prostate infection. These infections are more serious, as they can travel through the blood stream, causing urosepsis. Urosepsis makes individuals very ill and even critically ill. Individuals desire a device and method that eliminates the problem of a UTI going unnoticed and undetected.

Commonly, for detecting a UTI, a urine test is performed in a laboratory in which a urine sample is examined under a microscope for bacteria or white blood cells indicating a potential infection. Collecting a urine sample and providing to a laboratory for examination is both time consuming and costly for users. In some cases, the specimen may be collected using an invasive procedure called catheterization, which may be required to detect the presence of a UTI. Individuals desire a device and method of easily identifying a UTI without having to visit a doctor, emergency room and/or sending samples to a laboratory.

Therefore, there exists a long-felt need in the art for a device that can easily identify the presence of a potential urinary tract infection (UTI). There is also a long-felt need in the art for a device that provides potential UTI information to a user without having to visit a doctor. Additionally, there is a long-felt need in the art for a device that provides potential UTI information to a user without having to get a urine sample tested, or without having to submit to costly medical procedures. Moreover, there is a long-felt need in the art for a specialized device that reduces costs associated with UTI testing and that detects a UTI in a non-invasive manner. Further, there is a long-felt need in the art for a device that offers a method for users to quickly identify the presence of a UTI as quickly as possible. Furthermore, there is a long-felt need in the art for a UTI detection device that can be continuously used for long periods for detecting UTI. Finally, there is a long-felt need in the art for a UTI detection device that is comfortable to use, cost-effective, non-invasive, and quickly identifies the UTI.

The subject matter disclosed and claimed herein, in one embodiment thereof, comprises a non-invasive UTI detection device useful in detecting a possible urinary tract infection (UTI) easily in a cost-effective manner. The device is designed to be releasably placed inside an undergarment as an undergarment liner and is positioned on the crotch region of the undergarment for receiving urine thereon. The device features a color indicator strip and an opposite adhesive layer, the color indicator strip is formed of UTI detection reagents for detecting a UTI within 1-3 minutes by detecting presence of leukocytes, hemoglobin, and nitrates for indicating possibility of a UTI, the adhesive layer is configured to releasably adhere to a fabric, the color indicator strip is configured to change to red color for indicating definite UTI, green color for indicating no UTI, and yellow or orange color to indicate a possibility of a UTI when urine comes in contact with the strip.

In this manner, the UTI detection device of the present invention accomplishes all of the forgoing objectives and provides users with a non-invasive device that indicates possibility of a UTI in a cost-effective and convenient manner without requiring a user to go to a doctor or submit a specimen to a laboratory. The device remains secure inside the undergarment when worn and a color change of the device easily indicates the possibility of a UTI.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a non-invasive pad device useful in detecting a possible urinary tract infection (UTI). The device is designed to be releasably placed inside an undergarment as an undergarment liner, such that the device is positioned on the crotch region of the undergarment. The device features a color indicator strip layer and an opposing adhesive layer, the color indicator strip is formed of a UTI detection reagents for detecting a UTI within several minutes (i.e., 3 min.), the adhesive layer is configured to releasably adhere to a fabric, the color indicator strip is configured to change to a red color for indicating definite UTI, a green color for indicating no UTI, and a yellow or orange color to indicate a possibility of a UTI when urine comes in contact with the strip. The device is configured to detect presence of leukocytes, hemoglobin, and nitrates for indicating possibility of a UTI.

In yet another embodiment, a urinary tract infection (UTI) detection strip device is disclosed. The UTI detection strip is designed to releasably adhere to a crotch region inside of an undergarment. The strip has a leukocytes and nitrite test pad in the form of a color indicating strip, the strip is configured to change color when urine comes in contact with the strip, wherein a red color of the test pad indicates definite UTI, a green color indicates no UTI, and a yellow or orange color indicates a possibility of a UTI or dehydration.

In yet another embodiment, the UTI detection strip device is disposable and is thrown away (i.e., discarded) once used.

In yet another embodiment, the color indicating strip changes to red color upon detecting at least 100,000 leukocytes, hemoglobin, and nitrates for reliability and for removing the likelihood of false negatives.

In yet another embodiment, the UTI detection device conforms to meet “McGreer Criteria” for a UTI to be treated in a long-term care facilities.

In yet another embodiment, a method for non-invasive detection of a UTI is disclosed. The method includes the steps of providing a non-invasive UTI detection device, the device featuring a test pad for detecting leukocytes, hemoglobin, and nitrates, an adhesive layer for releasably adhering the device to the crotch region inside of an undergarment; adhering the device to the crotch region of an undergarment; urine comes in contact with the test pad; changing the color of the test pad to at least one of green, red or yellow, wherein green color indicates no detection of leukocytes, hemoglobin and nitrates; yellow color indicates the presence of less than 100,000 leukocytes and nitrates; and red color indicates the presence of at least 100,000 leukocytes and confirmed UTI.

In yet another embodiment, a kit for detecting a UTI is disclosed. The kit includes a plurality of UTI detection strips, each strip having a detection reagent; an instruction letter including instructions for using the strips, wherein a strip is adhered inside an undergarment on the crotch region allowing urine to contact thereon, the strip is configured to change colors to indicate possibility of a UTI or the detection of a UTI.

An object of the UTI detection strip of the present invention is to allow users to obtain information regarding a possible infection without having to visit a doctor, having an invasive procedure and without having lab work completed. Another object is to reduce costs associated with UTI testing, offering a method for users to quickly identify the presence of a UTI and get treatment as soon as possible.

Numerous benefits and advantages of this invention will become apparent to those skilled in the art to which it pertains upon reading and understanding of the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one potential embodiment of a UTI alert/detection device of the present invention in accordance with the disclosed architecture;

FIG. 2 illustrates a perspective view showing a bottom perspective view of the UTI alert/detection device of the present invention in accordance with the disclosed architecture;

FIG. 3 illustrates a perspective view showing the UTI detection undergarment liner device of the present invention positioned inside an undergarment in accordance with the disclosed architecture;

FIG. 4 illustrates a block diagram showing different colors indicated by the UTI detection device of the present invention for possibility of UTI in accordance with the disclosed architecture;

FIG. 5 illustrates a flow diagram illustrating steps performed to use the UTI detection device of the present invention for detecting possibility of a UTI in accordance with the disclosed architecture;

FIG. 6 illustrates a user wearing an undergarment with the UTI detection strip adhered therein in accordance with the disclosed architecture; and

FIG. 7 illustrates a UTI detection kit of the present invention in accordance with the disclosed architecture.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long-felt need in the art for a device that can easily identify the presence of a potential or definite urinary tract infection (UTI). There is also a long-felt need in the art for a device that provides potential UTI information to a user without having to visit a doctor. Additionally, there is a long-felt need in the art for a device that provides potential UTI information to a user without having to get a urine sample tested or undergoing costly medical procedures. Moreover, there is a long-felt need in the art for a specialized device that reduces costs associated with UTI testing and that detects a UTI in a non-invasive manner. Further, there is a long-felt need in the art for a device that offers a method for users to quickly identify the presence of a UTI as quickly as possible. Furthermore, there is a long-felt need in the art for a UTI detection device that can be continuously used or worn for long periods for detecting a UTI. Finally, there is a long-felt need in the art for a UTI detection device that is comfortable to use, cost-effective, non-invasive, and quickly able to identify the UTI.

The present invention, in one exemplary embodiment, is a urinary tract infection (UTI) detection strip device. The UTI detection strip is designed to releasably adhere to a crotch region inside of an undergarment for receiving a few drops of urine of a wearer of the undergarment. The strip has a leukocytes and nitrate test pad or test strip in the form of a color indicating or color changing strip, the strip is configured to change color when urine comes in contact with the strip, wherein a red color of the test pad indicates definite UTI, a green color indicates no UTI, and a yellow or orange color indicates a possibility of UTI or dehydration. The test pad is configured to detect leukocytes, hemoglobin, and nitrates in the urine for detecting a UTI.

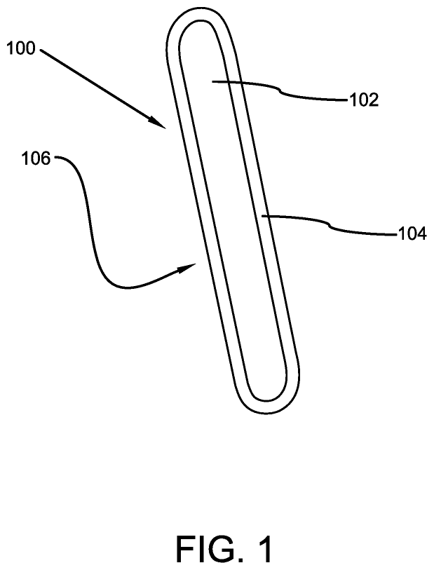

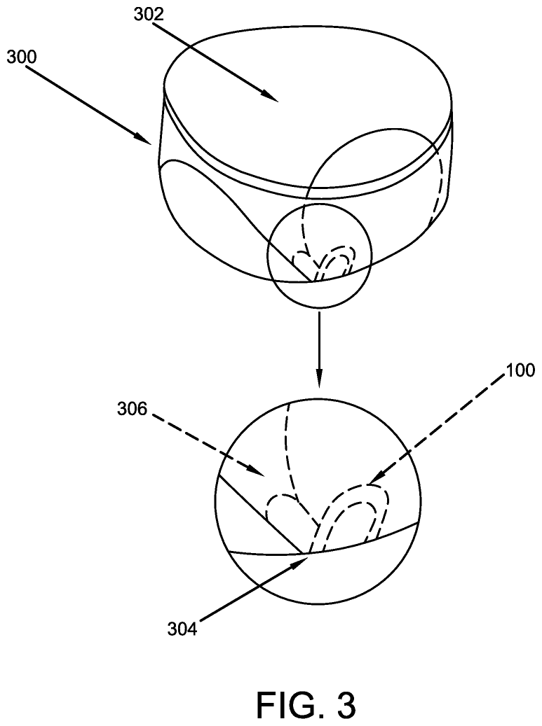

Referring initially to the drawings, FIG. 1 illustrates a perspective view of one potential embodiment of a UTI alert or detection device of the present invention in accordance with the disclosed architecture. The UTI alert/detection device 100 of the present embodiment is designed as a specialized device for easily detecting a possibility of a urinary tract infection (UTI) in a user in a non-invasive manner. The device 100 is in the form of a generally rectangular pad having a color indicator strip or color changing test pad 102 on a first surface 104 and an opposing adhesive surface 106 for releasably adhering the device 100 inside an undergarment as illustrated in FIG. 3. The device 100 is preferably designed and used as a liner for undergarments. The colored strip 102 is configured to detect laboratory values indicative of a urinary tract infection and based on the detected values, turns or changes to a specific color for indicating possibility of a UTI or definitive detection of a UTI.

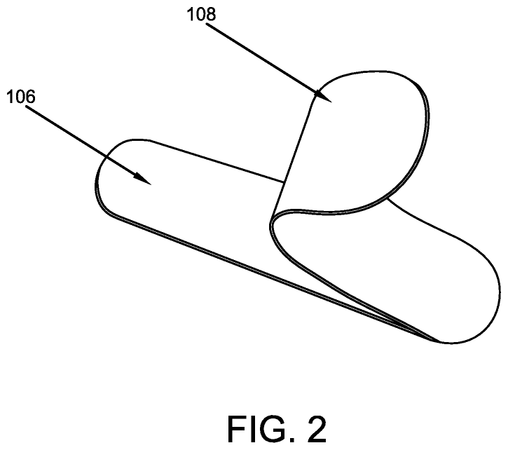

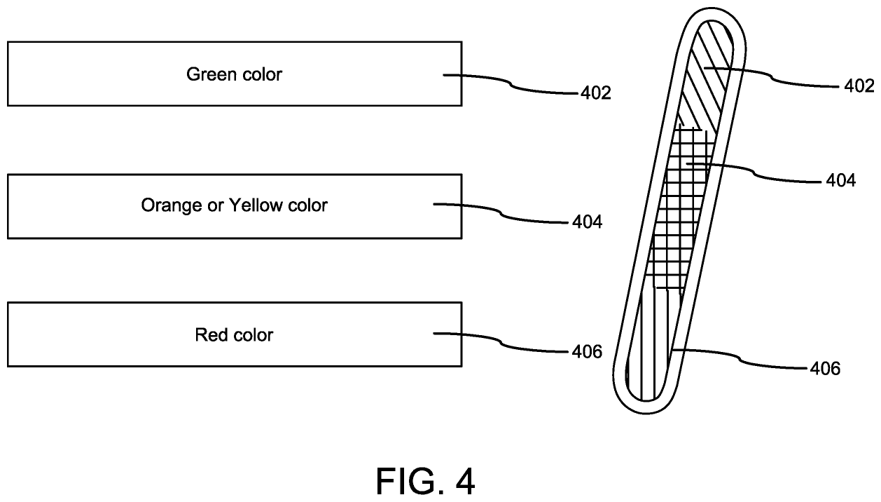

More specifically, the color indicating strip 102 is configured to analyze a few drops of urine dropped thereon and provide a corresponding color for possibility of a UTI as described in FIG. 4. The adhesive surface 106 is protected by a protection layer 108 as illustrated in FIG. 2 wherein the layer 108 is released to expose the adhesive surface 106 and to adhere the device 100 to a fabric such as an undergarment. The color indicating strip 102 detects the presence of leukocytes, nitrates, and other bacteria for detecting and indicating a UTI. The UTI detection pad or strip 100 is designed as a disposable device that can be worn and thrown away after a UTI detection occurs. The pad 100 may be available in a variety of sizes, types, and styles of undergarments such that drops of urine are directly dropped onto the color indicating strip 102 for detecting the UTI. In some embodiments, separate portions of the color indicating strip 102 can be used for separately testing leukocytes and nitrates.

FIG. 3 illustrates a perspective view showing the UTI detection undergarment liner device of the present invention positioned inside an undergarment in accordance with the disclosed architecture. As illustrated, the adhesive layer 106 is adhered to the inner surface 302 of the undergarment 300 preferably on both sides of the bottom fold line 304 of the crotch region 306. The liner device 100 is lightweight and has a thickness of less than 10 mm, less than 8 mm, less than 6 mm, or less than 4 mm in different embodiments of the present invention.

The device 100 is positioned such that urine of a wearer of the undergarment 300 can drop directly onto the color indicating strip 102 allowing device 100 to easily detect the possibility of the UTI in the wearer. The device 100 can be easily released from the crotch region 306 once used and different colors as described in FIG. 4 may indicate the level of a UTI.

FIG. 4 illustrates a block diagram showing different colors indicated by the UTI detection device of the present invention for possibility of a UTI in accordance with the disclosed architecture. The color indicator strip 102 on the pad 100 is configured to change colors to identify no UTI, potential UTI, and definite UTI. When urine contacts the color indicator strip 102 and the strip's color changes to green color 402, the strip 102 indicates absence of a UTI. When the strip's color changes to orange or yellow color 404, the strip 102 indicates a possibility of a UTI. The orange color 404 may also indicate dehydration in the user and the user may need to go for a urine test to ascertain a UTI.

A red color indication 406 of the strip 102 indicates a definite UTI and the need for an immediate treatment of the user/wearer. The red color indication 406 is a result of identification of leukocytes (over 100,000 units), hemoglobin, and nitrates, for a confirmation of a UTI. It should be appreciated that the red color 406 also conforms to “McGreer Criteria” for a UTI to be treated in long-term care. The red color 406 indicates the need for immediate use of antibiotics and a visit to a doctor for further treatment. The color indications may cover a portion of the strip 102 and the portions may or may not overlap each other.

It should be noted that the device 100 allows a user to easily identify a UTI without going to a doctor or laboratory. Further, the device 100 provides 99.9% accurate results for detection of leukocytes, hemoglobin, and nitrates.

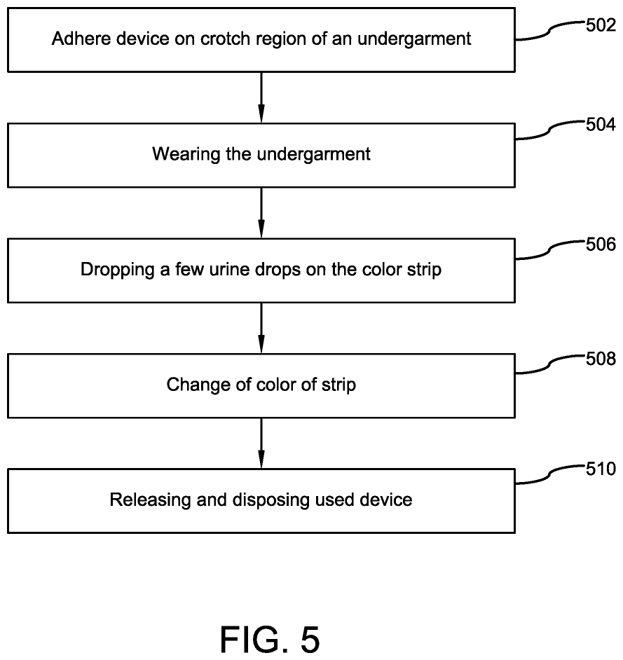

FIG. 5 illustrates a flow diagram illustrating steps performed to use the UTI detection device of the present invention for detecting possibility of a UTI in accordance with the disclosed architecture. Initially, the detection device is adhered on the crotch region inside of an undergarment (Step 502). Then, the undergarment is worn by a user who wants to check for the possibility of a UTI (Step 504). Thereafter, urine comes in contact with the color indicating strip/test pad of the device to wet the strip (Step 506). Color change of the strip is read by the user/wearer to determine the possibility of the UTI (Step 508). Finally, the strip is detached from the undergarment and is disposed of (Step 510).

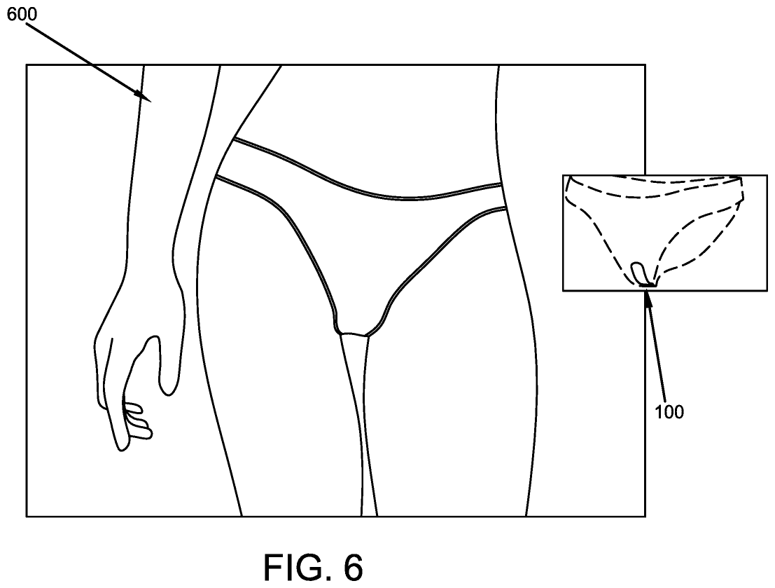

FIG. 6 illustrates a user wearing an undergarment with the UTI detection strip adhered therein in accordance with the disclosed architecture. Users of any age and gender can use the strip of the present invention and the strip device 100 is positioned on the crotch area such that drops of urine can easily drop onto the strip 102 of the device 100. The device 100 can be adhered as per preferences of the user 600 and color change in the strip may remain for at least 15-20 minutes.

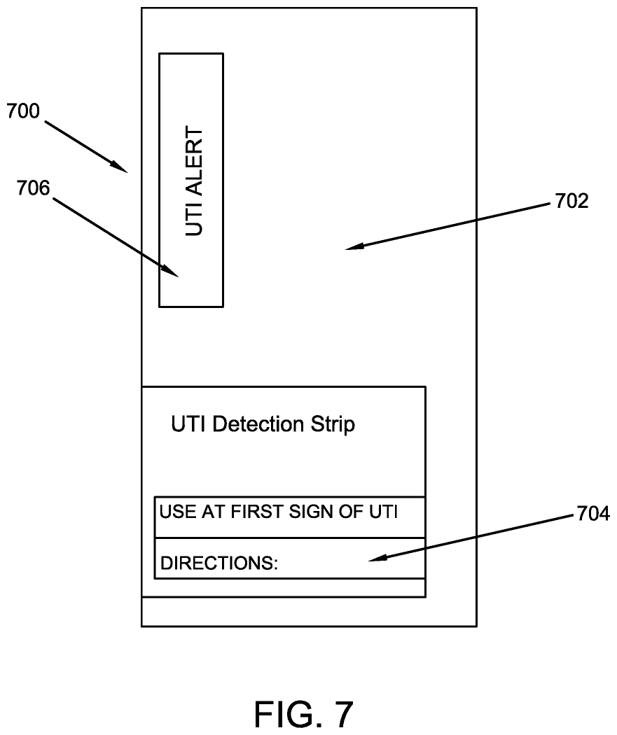

FIG. 7 illustrates a UTI detection kit of the present invention in accordance with the disclosed architecture. In the present embodiment, the UTI detection device 100 is commercially available as a kit 700 that may include a box 702 storing a plurality of UTI detection devices, an instruction manual 704 printed on the box 702, and one or more indicia 706. It should be noted that in some embodiments, the kit 700 may include, for example, 5, 10, 15, or 20 individual devices.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “UTI detection undergarment liner device”, “UTI alert/detection device”, “liner device”, “device”, and “UTI detection device” are interchangeable and refer to the non-invasive UTI detection device 100 of the present invention.

Notwithstanding the forgoing, the non-invasive UTI detection device 100 of the present invention can be of any suitable size and configuration as is known in the art without affecting the overall concept of the invention, provided that it accomplishes the above stated objectives. One of ordinary skill in the art will appreciate that the non-invasive UTI detection device 100 as shown in the FIGS. are for illustrative purposes only, and that many other sizes and shapes of the non-invasive UTI detection device 100 are well within the scope of the present disclosure. Although the dimensions of the non-invasive UTI detection device 100 are important design parameters for user convenience, the non-invasive UTI detection device 100 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A UTI detection device for detecting a possibility of a UTI, the UTI detection device comprising:
an indicator strip on a first surface; and
an adhesive on an opposing second surface;
wherein said adhesive releasably adheres said UTI detection device to an inner surface of an undergarment;
wherein said UTI detection device is a liner positioned between the undergarment and a wearer of the undergarment;
wherein said indicator strip having a detection portion for receiving drops of urine of the wearer for detecting the presence of at least one of leukocytes and nitrates; and
further wherein said detecting the presence of at least one of leukocytes and nitrates above a first threshold value indicates a possible UTI and above a second threshold value indicates a definitive UTI.

2. The UTI detection device of claim 1, wherein said UTI detection device having a selectively releasable protection layer to expose said adhesive layer prior to adhering inside the undergarment.

3. The UTI detection device of claim 1, wherein said detecting the presence of at least one of leukocytes and nitrates below said first threshold value indicates a lack of a UTI.

4. The UTI detection device of claim 1, wherein above said first threshold value changes said indicator strip to a first color.

5. The UTI detection device of claim 4, wherein said first color is yellow.

6. The UTI detection device of claim 5, wherein above said second threshold value changes said indicator strip to a second color.

7. The UTI detection device of claim 6, wherein said second color is red.

8. The UTI detection device of claim 6, wherein above said second threshold value is detection of leukocytes over 100,000 units.

9. The UTI detection device of claim 1, wherein below said first threshold value changes said indicator strip to a third color.

10. The UTI detection device of claim 1, wherein said UTI detection device is disposable.

11. A UTI detection device for detecting a possibility of a UTI, the UTI detection device comprising:
an indicator strip on a first surface; and
an adhesive on an opposing second surface;
wherein said adhesive releasably adheres said UTI detection device to an inner surface of an undergarment;
wherein said UTI detection device is a liner positioned between the undergarment and a wearer of the undergarment;
wherein said indicator strip having a detection portion for receiving drops of urine of the wearer for detecting the presence of at least one of leukocytes and nitrates;
wherein said detecting the presence of at least one of leukocytes and nitrates above a first threshold value indicates a possible UTI and above a second threshold value indicates a definitive UTI;
wherein above said first threshold value changes said indicator strip to a first color; and
further wherein above said second threshold value changes said indicator strip to a second color.

12. The UTI detection device of claim 11, wherein said UTI detection device having a selectively releasable protection layer to expose said adhesive layer prior to adhering inside the undergarment.

13. The UTI detection device of claim 11, wherein said detecting the presence of at least one of leukocytes and nitrates below said first threshold value indicates a lack of a UTI.

14. The UTI detection device of claim 11, wherein said first color is yellow.

15. The UTI detection device of claim 14, wherein said second color is red.

16. The UTI detection device of claim 11, wherein above said second threshold value is detection of leukocytes over 100,000 units.

17. The UTI detection device of claim 11, wherein below said first threshold value changes said indicator strip to a third color.

18. The UTI detection device of claim 11, wherein said third color is green.

19. A UTI detection device for detecting a possibility of a UTI, the UTI detection device comprising:
an indicator strip on a first surface; and
an adhesive on an opposing second surface;
wherein said adhesive releasably adheres said disposable UTI detection device to an inner surface of an undergarment;
wherein said disposable UTI detection device is a liner positioned between the undergarment and a wearer of the undergarment;
wherein said indicator strip having a detection portion for receiving urine of the wearer for detecting the presence of leukocytes; and
wherein said detecting the presence of leukocytes above a first threshold value changes said indicator strip to a first color and indicates a possible UTI;
wherein said detecting the presence of leukocytes above a second threshold value changes said indicator strip to a second color and indicates a definitive UTI; and
further wherein detecting the presence of leukocytes below said first threshold value changes said indicator strip to a third color and indicates a lack of a UTI.

20. The UTI detection device of claim 19, wherein above said second threshold value is detection of leukocytes over 100,000 units.
